(19) United States
(12) Patent Application Publication
(10) Pub. No.: US 2018/0136226 A1
(43) Pub. Date: May 17, 2018

(54) METHOD FOR DIAGNOSING AN INFLAMMATORY DISORDER

(71) Applicant: The Hospital for Sick Children, Toronto (CA)

(72) Inventor: Rae Yeung, Toronto (CA)

(21) Appl. No.: 15/527,523

(22) PCT Filed: Nov. 17, 2015
(86) PCT No.: PCT/CA2015/000576

§ 371 Date: Sep. 6, 2017

Related U.S. Application Data
(60) Provisional application No. 62/080,624, filed on Nov. 17, 2014.

(51) Int. Cl. G01N 33/68

(52) U.S. Cl. CPC G01N 33/6869; G01N 33/6893; G01N 2333/545; G01N 2800/328; G01N 2333/7155; G01N 2800/52; G01N 2800/7095; G01N 2333/54

(57) ABSTRACT

A method of diagnosing Kawasaki disease in a mammal is provided. The method comprises determining in a biological sample obtained from the mammal the expression level of at least IL-1RA, and optionally the level of one or more of, IL-Iβ, IL-18 and IL-18 BP, and diagnosing Kawasaki disease when the levels of these biomarkers is elevated in comparasion to febrile control levels.

### FIELD OF THE INVENTION

The present invention generally relates to diagnostic methods, and more particularly relates to a method of diagnosing an inflammatory disorder such as Kawasaki disease and predicting treatment response.

### BACKGROUND OF THE INVENTION

Kawasaki disease (KD) is a multisystem vasculitis that predominantly targets the coronary arteries in infants and young children. Coronary artery damage resulting from vascular inflammation in KD is the leading cause of acquired heart disease in developed nations.

Prolonged fever in a young child is the cardinal feature of KD. The syndrome complex associated with KD exemplifies the classic signs of inflammation, with redness, heat and swelling affecting multiple sites. The red skin, red eyes, red lips, red swollen hands/feet and swollen lymph nodes are non-specific findings and common signs of inflammation in many febrile illnesses of childhood. An important difference between KD and other febrile illnesses in children is that KD leads to coronary artery damage while common febrile illnesses do not. KD is defined by a constellation of non-specific symptoms and does not have a diagnostic test. Thus, one of the challenges of treating children with KD is early diagnosis, specifically distinguishing KD in a child from fever due to infections and other causes. Delays in patient identification and treatment lead to poor coronary outcome.

Thus, it would be desirable to develop a method of diagnosing KD that more accurately identifies KD as opposed to other febrile conditions.

### SUMMARY OF THE INVENTION

It has now been determined that inositol-triphosphate 3-kinase C regulates NLRP3 (nucleotide-binding domain and leucine-rich repeat containing (NLR) family, pyrin domain containing 3) activation via control of calcium mobilization, directing production of IL1β and IL18, and that regulation of calcium signaling is fundamental to the immunopathogenesis of KD.

Accordingly, in one aspect of the invention, a method of diagnosing Kawasaki disease in a mammal is provided comprising determining in a biological sample obtained from the mammal the expression level of at least IL-1RA, and optionally one or more of IL-1β, IL-18 and IL-18BP; comparing the expression level of IL-1RA, and optionally one or more of IL-1β, IL-18 and IL-18BP, to a febrile control value; and diagnosing the mammal with Kawasaki disease when the expression level of IL-1RA, and optionally the expression level of one or more of IL-1β, IL-18 and IL-18BP, is greater than the febrile control value.

In another aspect of the invention, a method of diagnosing Kawasaki disease in a mammal is provided comprising determining in a biological sample obtained from the mammal the expression level of at least IL-1RA, and optionally one or more of IL-1β, IL-18 and IL-18BP; and diagnosing the mammal with Kawasaki disease when the level of IL-1RA is at least about 470 pg/ml, and optionally, the level of IL-1β is at least about 5 pg/ml, the level the level of IL-18 is at least about 115 pg/ml, and/or the level of IL-18BP is at least about 5 ng/ml.

In another aspect, a method of predicting treatment response phenotype in a mammal with Kawasaki disease is provided, comprising determining in a biological sample obtained from the mammal the expression levels of IL-1βand IL-18, and determining that the mammal is at risk of being a non-responder to IVIG therapy when the level of IL-1β is greater than about 30 pg/ml, and/or the level of IL-18 is greater than about 95 pg/ml.

In a further aspect of the invention, a method of predicting treatment response phenotype in a mammal with Kawasaki disease is provided, comprising stimulating a biological sample obtained from the mammal with lipopolysaccharide and adenosine triphosphate, determining the expression levels of IL-1β or a related protein, and IL-18 or a related protein, in the sample, and determining that the mammal is at risk of being a non-responder to IVIG therapy when the level of IL-1β is greater than about 400 pg/ml, and/or the level of IL-18 is greater than about 50 pg/ml.

In a further aspect, a kit useful to diagnose Kawasaki disease in a mammal is provided. The kit comprises a biomarker-specific reactant for IL-1 receptor antagonist, and optionally one or more of IL-1β, IL-18 and IL-18 binding protein.

These and other aspects of the invention are described herein by reference to the following figures.

### BRIEF DESCRIPTION OF THE FIGURES

FIG. 1. Increased circulating IL-1β, IL-1RA, IL-18 and IL-18 BP in the acute phase of KD. (a) Box plots depicting concentrations of IL-1β and IL-18 together with their respective antagonists IL-1RA and IL-18BP, determined by ELISA, in serial plasma samples from children with KD during the acute (n=48) and convalescent (n=24) phases and in age-matched febrile controls (n=41). (b) Gene expression profile for IL-1β, its antagonist (IL-1RA) and IL-18, its antagonist (IL-18BP) in children with KD (n=171) during acute and convalescent phases of KD. (c) Gene expression profile for IL-1βcognate receptors (IL-1R1, IL-1R2) and IL-18 receptors (IL-18R1, IL-18RAP) from the same patient cohort. The box plots show the median (thick black line) within the box (25ₜₕ and 75ₜₕ percentiles) whose whiskers represent the 3_{rd} and 97ₜₕ percentiles. Linear generalized estimating equations were used to adjust for some repeated measures using SAS to calculate p-values (*p<0.001).

FIG. 2. Increased expression of NLRP3 inflammasome associated genes during acute KD. (a) Gene expression profile of inflammasome associated genes from serial samples from children with KD (n=171) during acute and convalescent phases of disease. The box plots show the median (thick black line) within the box (25ₜₕ and 75ₜₕ percentiles) whose whiskers represent the 3_{rd} and 97ₜₕ percentiles. *p<0.0001. (b) GeneMANIA functional association gene network for inflammasome associated genes. The physical interaction and co-expression between genes are expressed as pink and purple lines respectively, the thicker the line, the stronger the association. Gene names denoted in red, blue and black are up-regulated, down-regulated or unchanged, respectively during acute phase of KD.

FIG. 3. Absence of ITPKC alters intracellular calcium mobilization. BMDMs from C57BL/6 and ITPKC−/− were loaded with Fluo-4AM in Cₐ₂₊ free media and [Cₐ₂₊]ᵢ was measured using spinning disc confocal microscopy. Mean Fluorescence Intensity (Y-axis) of Fluo-4AM for cells in 5 independent fields (ROIs) over time (X-axis) (a) Media alone (b) 1.5 μM CaCl₂. (c) 1 mM ATP. Ionomycin (1 μM) was added at 30 minutes (arrow).

FIG. 4. ITPKC genotype dictates intracellular calcium mobilization and determines NLRP3 expression. (a) Mean fluorescence Intensity (MFI) of Fluo-4AM (Y-axis) acquired by Fluorescence-activated cell sorting (FACS) of EBV transfected B-cells from various ITPKC genotypes [GG (n=2), GC (n=2) and CC (n=2)] at rs28493229 plotted against time (X-axis). Graph represents average MFI of 4 experiments from each ITPKC genotypes. *p<0.001. (b) Representative FACS plots of [Cₐ₂₊]ᵢ (Fluo-4AM) over time (c) Protein expression of ITPKC protein assayed by immunoblot of EBV transfected B-cells from various ITPKC genotypes [GG (n=2), GC (n=2) and CC (n=2)], quantified by densitometry and expressed as arbitrary units (ITPKC protein expression normalized to GAPDH). (d) NLRP3 protein visualized by immunoblot of EBV transfected B cells from various ITPKC genotypes [GG (n=2), GC (n=2) and CC (n=2)] quantified by densitometry and expressed as arbitrary units (NLRP3 protein expression normalized to GAPDH) Western blots are representative of 3 repeats. (e) NLRP3 gene expression from children with KD (GG=111, GC=44, CC=5) (f) NLRP3 gene expression from EBV transfected B cells [GG (n=2), GC (n=2) and CC (n=2)] as determined by quantitative real time PCR.

FIG. 5: ITPKC genotype dictates intracellular calcium levels. (a) Mean fluorescence Intensity (MFI) of Fluo-4AM (Y-axis) acquired by FACS of EBV immortalized B-cells from three different ITPKC genotypes [GG (n=6), GC (n=5) and CC (n=7)] plotted against time (X-axis) from healthy controls. Graph represents average MFI of 2 experiments from each ITPKC genotypes. (b) Representative FACS plot showing [Cₐ₂₊]ᵢ (Fluo-4AM) over time acquired for 10 min for each ITPKC genotype.

FIG. 6: ITPKC regulates NLRP3 inflammasome activation and release of IL-1βand IL-18. (a) BMDMs from C57BL/6 or ITPKC_{-/-} were stimulated with LCWE (1 mg/ml) or LPS (100 ng/ml) and ATP (5 mM). NLRP3 protein was visualized by Immunoblot analysis, quantified by densitometry and expressed as arbitrary units (NLRP3 protein expression normalized to GAPDH). Western blots are representative of 3 separate experiments. (b) Coronary arteritis was induced by ip injection of LCWE as per protocol in 4-6 weeks old C57BL/6 (n=5) and ITPKC_{-/-} (n=5), together with PBS negative control injections. Serum IL-1βwas determined by ELISA at 48 hours. *p<0.0001. (c) BMDMs from C57BL/6 and ITPKC_{-/-} were stimulated with LCWE (0.1 μg/ml). IL-1βproduction was determined by ELISA. *p<0.0001. Data shown are representative of 3 experiments.

FIG. 7. ITPKC genotypes regulate production of IL-1β and IL-18 and influence treatment response in children with KD. (a) Children with KD [GG (n=101), GC (n=36), CC (n=10)] at rs28493229 response to IVIG treatment in the cohort are represented as % of nonresponders. (b) Maximum coronary artery dimensions expressed as body surface area normalized Z-scores from children with KD [GG (n=101), GC (n=36), CC (n=10)]. Box plots show the median (thick black line) within the box (25ₜₕ and 75ₜₕ percentiles) whose whiskers represent the 3_{rd} and 97ₜₕ percentiles. (c) IL-1β and (d) IL-18 plasma concentrations during the acute phase of KD [GG (n=2), GC (n=2) and CC (n=2)] as determined by ELISA. (e) IL-1β and (f) IL-18 production by PBMCs from affected children. PBMCs from GG (n=3) and CC (n=5) were stimulated with LPS and ATP. *p<0.0001.

FIG. 8. ITPKC regulates NLRP3 inflammasome activation. NLRP3 inflammasome activation is a two-step process with priming and an activation signal. Signal one triggers a pattern recognition receptor on APCs such as Toll like receptors (TLRs) to increase pro-IL-1β and pro-IL-18 via the NF-kB pathway. Signal two mediates the assembly and activation of the inflammasome, a large molecular platform composed of an NLR protein (such as NLRP3), the adaptor ASC and proinflammatory caspases (such as caspase 1). Activation of Caspase 1 cleaves pro-IL-1β and pro-IL-18 into their respective active cytokines. The following steps illustrate the contribution of ITPKC to the disease model: 1. ITPKC regulates phosphorylation of IP3 to IP4. Decreased ITPKC (knockout mice or humans with CC genotype) results in increased IP3, which binds to IP3R 2. Releasing of intracellular calcium [Cₐ₂₊]ᵢ from the endoplasmic reticulum (ER). 3. Increased [Cₐ₂₊]ᵢ increases NLRP3 expression and inflammasome activation leading to 4. Increased production of IL-1β and IL-18 from their pro-forms.

FIG. 9 illustrates the amino acid (A) and nucleic acid (B) sequences of IL-1β;

FIG. 10 illustrates the amino acid (A) and nucleic acid (B) sequences of IL-18;

FIG. 11 illustrates the amino acid (A) and nucleic acid (B) sequences of IL-1RA; and

FIG. 12 illustrates the amino acid (A) and nucleic acid (B) sequences of IL18BP .

### DETAILED DESCRIPTION OF THE INVENTION

A method of diagnosing Kawasaki disease is provided. The method comprises determining in a biological sample obtained from the mammal the expression level of at least IL1RA, and optionally, one or more of IL-1β, IL-18 and IL-18BP; and diagnosing the mammal with Kawasaki disease when the expression level of IL-1RA is at least about 470 pg/ml, and optionally, the level of IL-1β is at least about 5 pg/ml, the level of IL-18 is at least about 115 pg/ml, and/or the level of IL-18BP is at least about 5 ng/ml.

The term “Kawasaki Disease” refers to an inflammatory disorder which satisfies the following criteria as defined by the American Heart Association: prolonged fever (at least 5 days in duration) plus the presence of at least 4 of the following 5 principal features: 1) polymorphous skin rash; 2) bilateral nonexudative conjunctival injection; 3) oral-mucosal changes including erythematous, cracked lips, strawberry tongue and injection of the oral and pharyngeal mucosa; 4) extremity changes including erythema of the palms and/or soles of the feet, swelling of the hands and/or feet and periungual peeling of the fingers and/or toes in the sub-acute phase; and 5) cervical lymphadenopathy (>1.5 cm diameter−usually unilateral). Complete Kawasaki Disease is the presence of at least of the principal features, while less than 4 of the 5 principal diagnostic features constitutes incomplete Kawaski Disease. Kawasaki Disease can be divided into 3 phases: acute, subacute and convalescent. The acute phase is characterized by a multisystem vasculitis as described above. The subacute phase is characterized clinically by resolution of the fever, systemic symptoms and peeling of the skin of the fingers. During this phase, coronary artery lesions (CAL) are most commonly detected (4-6 weeks) and there are still persistent biochemical and hematologic markers of inflammation on standard laboratory testing typified by elevated platelet count and CRP. The convalescent phase is defined as the recovery period of the disease in which a patient is generally asymptomatic from an inflammatory standpoint and inflammatory markers and platelet count have returned to normal baseline levels.

The term “IL-1β ” or “interleukin-1β” refers herein to the mammalian cytokine and encompasses both human IL-1β, as depicted by NCBI accession no. NP000567, as well as functionally equivalent IL-1β of other mammalian species, for example, mouse IL-1β as depicted NCBI accession no. NP032387, as well as functionally equivalent variants or isoforms of an IL1β. Human IL-1β is encoded by mRNA as depicted by NCBI accession no. NM000576, and functionally equivalent mRNA, e.g. such as mRNA encoding IL-1β of other mammalian species, for example, mouse IL-1β mRNA as depicted by NCBI accession no. NM 008361; including one or more codon differences due to degeneracy in the genetic code; or encoding functionally equivalent IL-1β variants or isoforms.

The terms “IL-1R1” and “IL-1R2” refer herein to the mammalian receptors of IL1β. IL-1R1 encompasses the human receptor depicted by NCBI accession no. NP 000868, as well as functionally equivalent IL-1R1 of other mammalian species, for example, mouse IL-1R1 as depicted by NCBI accession no. NP 001116854, and functionally equivalent variants or isoforms of an IL-1R1. IL1-R2 encompasses the human receptor depicted by NCBI accession no. NP 001248348, as well as functionally equivalent IL-1R2 of other mammalian species, for example, mouse IL-1R2 as depicted by NCBI accession no. NP 034685, and functionally equivalent variants or isoforms thereof. Human IL-1R1 is encoded by mRNA as depicted by NCBI accession no. NM000877, and functionally equivalent mRNA, e.g. such as mRNA encoding IL-1R1 of other mammalian species, for example, mouse IL-1R1 mRNA as depicted by NCBI accession no. NM 001123382; including one or more codon differences due to degeneracy in the genetic code; or encoding functionally equivalent IL-1R1 variants or isoforms. Human IL-1R2 is encoded by mRNA as depicted by NCBI accession no. NM001261419, and functionally equivalent mRNA, e.g. such as mRNA encoding IL-1R2 of other mammalian species, for example, mouse IL-1R2 mRNA as depicted by NCBI accession no. NM 010555; including one or more codon differences due to degeneracy in the genetic code; or encoding functionally equivalent IL-1R2 variants or isoforms.

The term “IL-18” or “interleukin-18”, also known as interferon-gamma inducing factor, refers to the mammalian cytokine and encompasses both human IL-18, as depicted by NCBI accession no. NP001230140, as well as functionally equivalent IL-1 of other mammalian species, for example, mouse IL-18 as depicted NCBI accession no. NP032386, and any functionally equivalent variants or isoforms of an IL-18. Human IL-18 is encoded by mRNA as depicted by NCBI accession no. NM001243211, and functionally equivalent mRNA, e.g. such as mRNA encoding IL-18 of other mammalian species, for example, mouse IL-18 mRNA as depicted by NCBI accession no. NM 008360; including one or more codon differences due to degeneracy in the genetic code; or encoding functionally equivalent IL-18 variants or isoforms.

The terms “IL-18R1” and “IL-18RAP” refer to the receptors of IL-18. IL-18R1 encompasses the human receptor depicted by NCBI accession no. NP 001269328, as well as functionally equivalent IL-18R1 of other mammalian species, for example, mouse IL-18R1 as depicted by NCBI accession no. NP 001155314, and functionally equivalent variants or isoforms of an IL-18R1. IL-18RAP encompasses the receptor depicted by NCBI accession no. NP 003844, as well as functionally equivalent IL-18RAP of other mammalian species, for example, mouse IL-18RAP as depicted by NCBI accession no. NP 043683, and functionally equivalent variants or isoforms thereof. Human IL-18R1 is encoded by mRNA as depicted by NCBI accession no. NM001282399, and functionally equivalent mRNA, e.g. such as mRNA encoding IL-18R1 of other mammalian species, for example, mouse IL-18R1 mRNA as depicted by NCBI accession no. NM 001161842; including one or more codon differences due to degeneracy in the genetic code; or encoding functionally equivalent IL-18R1 variants or isoforms. Human IL18RAP is encoded by mRNA as depicted by NCBI accession no. NM003853, and functionally equivalent mRNA, e.g. such as mRNA encoding IL-18RAP of other mammalian species, for example, mouse IL-18RAP mRNA as depicted by NCBI accession no. NM 010553; including one or more codon differences due to degeneracy in the genetic code; or encoding functionally equivalent IL-18RAP variants or isoforms.

The term “IL-1 receptor antagonist” or “IL-1RA” refers to a protein that naturally inhibits IL-1β. It is encoded by the IL1RN gene in humans. IL-1RA encompasses both human IL-1RA, as depicted by NCBI accession no. NP000568, as well as functionally equivalent IL1RA of other mammalian species, for example, mouse IL-1RA as depicted NCBI accession no. NP001034790, and functionally equivalent variants or isoforms of an IL-1RA. Human IL-1RA is encoded by mRNA as depicted by NCBI accession no. NM000577, and functionally equivalent mRNA, e.g. such as mRNA encoding IL-1RA of other mammalian species, for example, mouse IL-1RA mRNA as depicted by NCBI accession no. NM 001039701; including one or more codon differences due to degeneracy in the genetic code; or encoding functionally equivalent IL-1RA variants or isoforms.

The term “IL-18 binding protein” or “IL-18BP” refers to a protein that naturally inhibits IL-18. It is encoded by the L18BP gene in humans. IL-18BP encompasses both human IL-18BP, as depicted by NCBI accession no. NP001034748, as well as functionally equivalent IL-18BP of other mammalian species, for example, mouse IL-18BP as depicted NCBI accession no. NP034661, and any functionally equivalent variants or isoforms of an IL-18BP. Human IL-18BP is encoded by mRNA as depicted by NCBI accession no. NM001039659, and functionally equivalent mRNA, e.g. such as mRNA encoding IL-18BP of other mammalian species, for example, mouse IL-18BP mRNA as depicted by NCBI accession no. NM 010531; including one or more codon differences due to degeneracy in the genetic code; or encoding functionally equivalent IL-18BP variants or isoforms.

The term “functionally equivalent” as used herein is meant to refer to forms of a compound, e.g. such as IL-1β, IL-18, IL-1RA and IL-18BP, including all mammalian forms from different species, and isoforms, variants or mutants of any of these, that possesses the same or similar function and/or activity, i.e. at least about 30% of the activity of the parent compound, and preferably at least about 50% or greater of the activity of the parent compound.

The term “mammal” is used herein to refer to both human and non-human mammals including domestic animals, e.g. cats, dogs and the like, livestock and undomesticated animals.

In a first step of the present method, a biological sample is obtained from a mammal. The term “biological sample” is meant to encompass any mammalian sample that contains IL-1RA, and optionally, one or more of the biomarkers, IL-1β IL-18 or IL-18BP, and/or related proteins, e.g. related proteins, such as isoforms, variants or receptors as described above, that may be indicative of the level or concentration of one of IL-1β, IL-18, IL-1RA or IL-18BP, in the sample. For example, levels of IL-1R1 and IL-1R2, the receptors for IL-1β, are also upregulated with upregulation of IL-1β. Similarly, levels of IL-18R1 and IL-18RAP, the receptors for IL-18, are upregulated with the upregulation of IL-18. Thus, the levels of one or more of the receptors may be used instead of the cytokine or in conjunction therewith. Suitable biological samples include, for example, whole blood, serum, plasma, urine, cerebrospinal fluid, ascitic fluid, lacrimal fluid, bone marrow or derivatives of any of these. The sample is obtained from the mammal in a manner well-established in the art.

Once a suitable biological sample is obtained, it is analyzed for the expression level of IL-1RA, and optionally, one or more of the biomarkers, IL-1β, IL-18 or IL-18BP. If appropriate, a related protein indicative of the level of one of IL-1β, IL-18, IL-1RA or IL-18BP, such as IL-1R1 or IL-1R2, or IL-18R1 or IL-18RAP, may be analyzed in place of the protein to which it is related. This determination may be accomplished using various methods established in the art, for example, by Enzyme Linked ImmunoSorbent Assays (ELISAs) or Enzyme ImmunoAssay (EIA). In this assay, the biomarker to be analyzed is complexed with a reactant specific for the biomarker, such as an antibody which is linked (either before or following formation of the complex) to an indicator, such as an enzyme. Examples of antibodies for IL-1β include antibodies from Abcam (e.g. ab9722), Santa Cruz Biotechnology (e.g. (H-153): sc-7884) and R&D Systems; antibodies for IL-18 include antibodies from LifeSpan Biosciences, Inc. (e.g. LS-B2809) and R&D Systems; antibodies for IL-1RA include antibodies from Abeam (e.g. ab2573), Santa Cruz Biotechnology (e.g. (H-110): se-25444) and R&D Systems; and antibodies for IL-18BP include antibodies from Santa Cruz Biotechnology (e.g. H-61), Novus Biologicals (e.g. NB200-201) and R&D Systems. Detection may then be accomplished by incubating this enzyme-complex with a substrate for the enzyme, for example, that produces a detectable product. The indicator may be linked directly to the reactant (e.g. antibody) or via a linker, such as a secondary antibody that recognizes the first or primary antibody, or a protein such as streptavidin, if the primary antibody is biotin-labeled. Examples of suitable enzymes include, but are not limited to, horseradish peroxidase (HRP), alkaline phosphatase (AP), ß-galactosidase, acetylcholinesterase and catalase. A large selection of substrates is available for performing the ELISA with an HRP or AP conjugate. Useful substrates depend on the level of detection required and the detection instrumentation used, e.g. spectrophotometer, fluorometer or luminometer.

A convenient method by which multiple biomarker levels in a sample may be determined (e.g. IL-1RA, and optionally, one or more of the biomarkers, IL-1β, IL-18 or IL18BP) utilizes, for example, biochip array technology. Generally, biochip arrays provide a means to simultaneously determine the level of multiple biomarkers in a sample. These arrays may be based on ELISA principles of sandwich or competitive immunoassays and, thus, the biochip provides a reaction platform including biomarker-specific antibodies attached at pre-defined sites on the surface, e.g. multiplex assay (Luminex®). An amount of biological sample in the range of about 80-120 μl is generally used to determine biomarker levels utilizing a biochip.

Other methods of detecting the presence of the target proteins (e.g. IL-1RA, and optionally, IL-1β, IL-18 and/or IL-18BP, or related proteins) may also be utilized as would be appreciated by one of skill in the art, including for example, mass spectrometry, gel electrophoresis as used in Western Blot techniques and other methods used in clinical chemistry.

In another embodiment, the expression level of protein biomarkers in a biological sample from a mammal may be determined based on the levels of nucleic acid (i.e. DNA or mRNA transcript encoding the target protein biomarkers in the biological sample. Methods of determining DNA or mRNA levels are known in the art, and include, for example, PCR-based techniques (such as RT-PCR), microarrays, the Nanospring nCounter™ gene expression system using color-coded probe pairs and Northern or Southern blotting techniques which generally include the application of gel electrophoresis to isolate the target nucleic acid, followed by hybridization with specific labeled probes. Probes for use in these methods can be readily designed based on the known sequences of genes encoding the protein biomarkers, as well as the known amino acid sequence of the target biomarkers. Suitable labels for use are well-known, and include, for example, fluorescent, chemiluminescent and radioactive labels.

Once the concentration or level of at least IL-1RA, and optionally the level of one or more of IL-1β, IL-18 and/or IL-18BP, in the sample is determined, it may be compared to a febrile control level, i.e. the level of the biomarker in an age-matched non-KD mammal with fever. An increase in the level of at least IL-1RA, and optionally one or more of IL-1β, IL-18 and/or IL-18BP, to a febrile control value is sufficient to be indicative of KD. For example, increased levels of at least IL-1RA, and optionally IL-1β, IL-18 and/or IL-18BP, of at least about 10%, preferably 20% or greater, e.g. 30%, 40%, 50% or greater, as compared to a febrile control level is indicative of KD. More particularly, a level of IL-1RA of at least about 470 pg/ml, and optionally, a level of IL-1β of at least about 5 pg/ml, a level of IL-18 of at least about 115 pg/ml, and a level of IL-18BP of at least about 5 ng/ml, is indicative of KD.

As one of skill in the art will appreciate, the ratio of certain biomarkers may also be indicative of KD. For example, the ratio of IL-1β to IL-1RA levels may be determined and compared to the ratio in a febrile control, and a change in the ratio of at least about 10% may be indicative of KD. Likewise, the ratio IL-18 to 1L-18BP levels may be determined and compared to the ratio in a febrile control, and a change in the ratio of at least about 10% may be indicative of KD.

The present biomarkers may also be useful to identify disease phenotype, specifically treatment response phenotype, and thus, may be used to predict or identify patients that have KD but that will not respond, or have a high probability of non-response, to the standard of care (intravenous immunoglobulin (IVIG) therapy) for KD, i.e. identify candidates at high risk for treatment failure and thus requiring intensive/alternative therapy such as anti-IL-1 therapy. In this regard, the levels of IL-1β and IL-18 are measured in a biological sample from a patient. A level of IL-1β of greater than about 30 pg/ml (e.g. at least about 30-40 pg/ml), and/or a level of IL-18 of greater than 95 pg/ml (e.g. at least about 100 pg/ml, 200 pg/ml, 300 pg/ml, or greater) is indicative of the ITPKC genotype, i.e. the presence of the CC allele genotype, and a high probability of being a non-responder to IVIG therapy. The CC allele genotype may be used itself as a marker of a non-responder to IVIG therapy, or may be used in conjunction with one or both of IL-1β and IL-18, to identify non-responders.

In another embodiment, treatment response phenotype in a mammal with Kawasaki disease may be predicted by stimulating a biological sample obtained from the mammal with lipopolysaccharide (LPS) and adenosine triphosphate (ATP), with amounts of each which cause such stimulation, using well-established methods. Following such stimulation, the expression levels of IL-1β or a related protein, and IL-18 or a related protein, in the sample is determined using techniques as above-described (e.g. ELISA). A mammal is at risk of being a non-responder to IVIG therapy when the level of IL-1β is determined to be greater than about 400 pg/ml, and/or the level of IL-18 is determined to be greater than about 50 pg/ml.

A kit useful to diagnose Kawasaki disease in a mammal is provided. The kit comprises a biomarker-specific reactant for at least IL-1 receptor antagonist, and optionally for one or more of IL-1β, IL-18 and IL-18 binding protein. The biomarker-specific reactant for each biomarker may be an antibody specific to each. Each of the reactants is associated with an indicator (e.g. an enzyme, such as horseradish peroxidase (HRP), alkaline phosphatase (AP), ß-galactosidase, acetylcholinesterase and catalase) which is capable of yielding a detectable product that is indicative of the concentration of the cytokine in the biological sample, for example, by reacting with a substrate to produce a detectable product. The kit may additionally include instructions indicating the levels of the biomarkers that are indicative of KD as previously described.

Embodiments of the present invention are illustrated in the following specific example which is not to be construed as limiting.

### EXAMPLE 1

### Methods

Patient Cohorts. Children were included in these studies if they satisfied the American Heart Association (ABA) diagnostic criteria for Kawasaki Disease (as described by Newburger J W et at. Circulation. 2004; 110(17):2747-2771). Informed consent for participation was obtained from parents and informed consent or assent was obtained from patients as appropriate. The patient cohorts consisted of 185 children with KD. The median age at diagnosis was 33 months, 90% of subjects had complete KD with median fever duration of 7.1 days, 61% of the cohort were boys and all received IVIG therapy. The age-matched febrile controls were diagnosed with bacterial or viral infections. Healthy control DNA and EBV transformed cell lines were obtained from a healthy adult population-base. Participants were healthy adult volunteers who provided information on demographics, ethnicity, and completed a detailed questionnaire for major diseases including autoimmune, cardiovascular, respiratory, genetic/metabolic, and allergic diseases. Their respective institutional research ethics boards approved all studies.

Clinical and laboratory data. Detailed demographic, clinical, and laboratory data were captured at diagnosis (pre-treatment) and at 6 months. Clinical data were collected prospectively using standardized clinical reporting forms, which captured all key features in the AHA diagnostic criteria and included standard laboratory markers and standardized echocardiographic imaging of the heart and coronary arteries. Coronary dimensions were converted to body surface area normalized z-scores as per protocol (de Zorzi et al. J Pediatr. 1998; 133(2):254-258).

Biologic sample collection. Peripheral blood samples were collected and processed as per standardized protocol at each study visit and stored locally at −80° C., then transported to the central analysis sites for cytokine analysis and cellular assays or gene expression microarray.

Mice. Four week old C57BL/6 mice were purchased from The Jackson Laboratory (Bar Harbor, Me.). ITPKC knockout mice were generated by S. Schurmans (Pouillon et al. Nat Immunol. 2003; 4(11):1136-1143). Both wild type C57BL/6 and ITPKC_{-/-} mice were housed under specific pathogen-free conditions at the University Health Network (Toronto, Canada). All animal studies were performed according to guidelines and procedures approved by Animal Care Committee at both the Hospital for Sick Children Research Institute, and the University Health Network (Toronto, Canada).

In vivo studies. Four week old C57BL/6 or ITPKC−/− mice were injected with either PBS alone, or Lactobacillus casei cell wall extract (LCWE) (1 mg/mouse) was administered intraperitoneally as per protocol. Animals were sacrificed at 48 hours, blood collected, serum extracted, aliquoted and stored at −80° C. until use. LCWE was prepared as previously described (Lehman et al. Arthritis Rheum. 1985; 28(6):652-659).

Cell Cultures. EBV-transformed cell lines from affected children (Franco et al. Autoimmunity. 2014; 47(2):95-104) and from healthy controls were obtained and maintained in RPMI-1640 (VWR International, Missisauga, ON, Canada) supplemented with 10% FBS, 1 mM Sodium pyruvate, 0.1 mM non-essential amino acid, 50 mM 2-ME, 2 mM L-glutamine and 10 mM HEPES, 100 U/ml penicillin and 100 mg/ml streptomycin (Life Technologies, Burlington, ON, Canada) (complete RPMI).

Bone marrow derived macrophages (BMDMs). BMDMs were obtained by differentiating bone marrow progenitors from the tibia and femur of 8-12 week old wild type C57BL/6 or ITPKC−/− mice in complete RPMI containing 20 ng/ml of M-CSF (Sigma Aldrich, Mo., USA), for 7 days. BMDMs were then re-plated in 24 well plates at 2.5×10₅ cells/ml (VWR International) 1 day before experimental assays.

Inflammasome activation, EBV-transformed B-cells were cultured in complete RPMI-1640 at 2×106 cells/ml in 48-well plate, in the presence of absence of either 100 ng/ml LPS, 5 mM ATP or LPS+ATP (Sigma Aldrich). Following an incubation period of 18 hours, supernatants were collected and IL-1β and IL-18 assayed by ELISA according to the manufacturer's instructions (eBiosciences, San Diego, Calif.). Cell pellets were collected, and stored at −80° C. until used in western blotting as described below. In all cases, ATP was added only during the last 30 minutes of incubation. In some experiments, EBV transformed cells were similarly stimulated; cell pellets were harvested for RNA extraction and NLRP3 mRNA expression by qRT-PCR.

In experiments where PBMC from affected children were used, the cells were cultured for 5 hours at 2×10⁶ cells in 48-well plate, in either complete RPMI alone, or complete RPMI containing 400 ng/ml LPS. ATP was added at a final concentration of 5 mM during the last hour of incubation. Supernatants were then collected, and assayed for IL1β and IL18 by ELISA. BMDMs from either wild type C57BL/6 or ITPKC−/− were cultured at 2.5×10⁵ cells/ml in 24 well plates in complete RPMI alone, LCWE (0.1 mg/ml ), or LPS (1 mg/ml ) (Sigma Aldrich) for 18 hours. ATP (5 mM) (Sigma Aldrich) was added during the last 45 min of incubation for activation of inflammasomes. Supernatants and cell lysates were collected, and assayed for IL18β and IL18 by ELISA, and for NLRP3 protein by western blotting, respectively.

Gene Expression Microarray. Illumina gene expression microarray platforms were used in this study. The detailed protocol was as previously published (Hoang et al. J Virol. 2010; 84(24):12982-12994). One-color array technology on the Illumina microarray platform (Illumina Inc, San Diego, Calif., USA) was used to analyze gene expression. In brief, whole-blood (2.5 ml) was collected directly into PAXgene RNA tubes (Qiagen, Sussex, UK). RNA extraction was performed using Paxgene RNA kits (Qiagen). Biotinylated amplified cRNA was generated by in vitro transcription (IVT) technology using Illumina® TotalPrep™ RNA Amplification Kit (Ambion, Inc., Austin, Tex., USA) according to the manufacturer's instructions. After purification, 2 μg of cRNA was hybridized to an Illumina HumanRef-12 V4 BeadChip (containing probes for more than 47,000 gene transcripts) at 55° C. for 18 hours following the manufacturer's instructions (Illumina, Inc., San Diego, Calif., USA). This was followed by washing, blocking and streptavidin-Cy3 staining steps. Finally, the chip was scanned with an Illumina Bead Array Reader confocal scanner and checked using Illumina QC analysis. Background subtracted raw gene expression intensity data was exported from Genomestudio and used for further analysis. Data was quantile normalized using Lumi R package (www.bioconductor.org) before log 2 transformation was performed. Finally, only genes that were detected (detection P value ≤0.001) in at least one sample were used for downstream analysis.

Quantitative RT-PCR analysis. Cells were lysed with TRIzol® reagent (Life Technologies), and total RNA were isolated with a standard chloroform extraction method. cDNA was synthesized using the GeneAmp® RNA PCR kit and murine leukemia virus reverse transcriptase (Life Technologies). cDNA was then amplified by RT-PCR following the manufacturers protocol using predesigned TagMan® primers and probe set specific for human NLRP3 (Assay ID# Hs00918082_ml) and human GAPDH on ABI Prism 7900 HT (Life Technologies). Relative gene expression is presented as the ratio of gene of interest quantity to GAPDH quantity for each cDNA sample. Data were analyzed using Sequence Detection Software (v.2.2.2, Life Technologies).

Immunoblots. Proteins from lysates of BMDMs and EBV transformed B-cells were extracted in Radio-Immunoprecipitation (RIPA) Buffer (Sigma Aldrich) containing Halt™ protease and phosphatase inhibitor cocktail (Fisher Scientific, Napean, ON, Canada). Immunoblots were prepared with Bolt® Bis-Tris Plus Gel (Life Technologies), and western blot analysis was carried out according to standard protocols, with antibodies specific for rabbit polyclonal raised against human NLRP3 (1:6500, se-66846, Santa Cruz Biotechonology Inc., Santa Cruz, Calif.) or rabbit polyclonal raised against human ITPKC (1:450, PAS-26334, Fisher Scientific). GAPDH was used as loading control (AM4300, Life Technologies). Relative protein levels were normalized to GAPDH as determined by densitometry using Image J software (1.8v, NIH)

Quantification of secreted cytokines. All ELISAs were carried out as per manufacturers' protocols. The following ELISAs were used in this study: Mouse ILβ, IL18 in cell culture supernatants or serum was measured using mouse-IL1β Ready-SET-Go!® and mouse-IL18 Platinum ELISA kits (eBiosciences). Human IL1β, IL18, IL1RA, IL18BP in plasma of children were measured using the following ELISA kits: Human IL1β Ready-SET-Go!®, Human-IL18 Platinum, Human IL1RA (eBiosciences), and Human IL18 BP (Abeam Inc, Toronto, ON, Canada).

Calcium analysis by confocal microscopy. BMDMs were plated on 4-chambered glass dishes (Fisher Scientific) at 0.1×10⁶ cells per chamber. Cells were loaded with Fluo-4/AM (Life Technologies) in Ca²⁺ free DMEM media (Life Technologies) supplemented with 10% FBS, 1 mM Sodium pyruvate, 0.1 mM non-essential amino acid, 50 mM 2-ME, 2 mM L-glutamine and 10 mM HEPES (complete-DMEM) and maintained in 37° C. Live images of untreated cells were taken at t=0. Cells were then treated with 1 mM ATP or 1.5 mM CaCl₂ in Ca²⁺ free DMEM at t=0. Cells were imaged for 40 min with acquisition at 15 sec intervals. Ionomycin (1 mM) (Sigma Aldrich) was added at the end of 30 min to the medium, and cells were imaged for the remaining time. Images were acquired using Olympus 1X81^{z} motorized inverted fluorescence microscope with a Hamamatsu C9100-13 black-thinned EM-CCD camera and Yokogawa CSU X1-spinning disc confocal imaging system using the 488-nm laser and emission in the range of 500-600 (Carl Zeiss, Toronto, ON, Canada). Images were analyzed using Volocity software (Perkin Elmer, Woodbridge, ON, Canada). Absolute intensity for cells in 5 independent fields at different time points was obtained and the average was displayed as the mean fluorescence intensity (MFI) of all cells in the fields.

Calcium analysis by Fluorescence Activated Cell Sorting (FACS). EBV transfected B cells (5-8×10⁶ cells) were loaded with Fluo-4/AM and Fura Red (Life Technologies) loading dyes in Ca²⁺ free complete DMEM media and incubated at 37° C. for 35 min. The cells were rested at room temperature for 20 min before diluting the loaded cells to 3×10⁶ cells/ml in Ca²⁺ free DMEM media supplemented with 10% FBS, 1 mM Sodium pyruvate, 0.1 mM non-essential amino acid, 5 mM 2-ME, 2 mM L-glutamine and 10 mM HEPES. [Ca^{2+]}i mobilization was acquired for continuous 10 minutes using BD™ LSR II Analyzer (BD Biosciences, Mississauga, ON, Canada). To flux Ca²⁺, Ionomycin (1 mM) was added at 1 min during the acquisition. Peak MFI of Fluo-4AM per minute is represented over time. Data were analyzed using FlowJo v9 (Tree star, OR, USA) software.

Functional gene prediction. GeneMANIA program (http://www.genemania.org) was used with the following query genes in humans: NLRP3, NLRC4, NLRP12, IL1β and IL18. The network was analyzed for physical interactions and co-expression of genes to the query genes. Based on the microarray data, expression of genes during the acute phase of KD were compared with convalescent phase of gene expression and represented as a network with up-regulated genes (red), down regulated genes (blue) and unaltered genes (black).

Genotyping. For genotype, DNA was extracted as previously described (Burns et al. Genes and Immunity. 2005; 6(5):438-444) from affected children or healthy controls and PCR was performed for polymorphism (rs28493229; Applied Biosystems, C_25932098_10) with 20 ng of DNA following manufacturer's instruction.

Statistical analyses. To compare febrile controls, and patients with acute and convalescent KD (paired data), linear generalized estimating equations were used to adjust for some repeated measures using Statistical Analysis Software (SAS). t-test or two-way ANOVA were used as appropriate for analysis of both in vivo or in vitro experiments in mice and man.

### Results

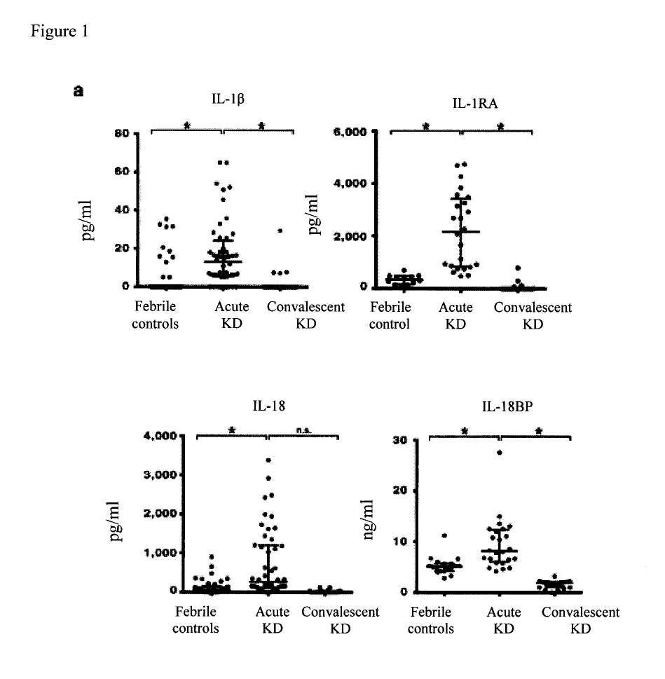

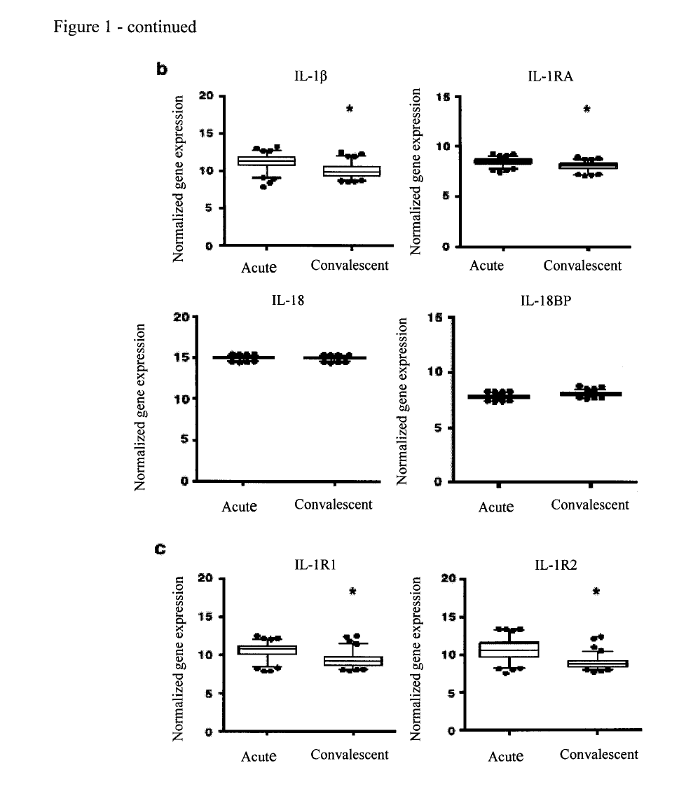

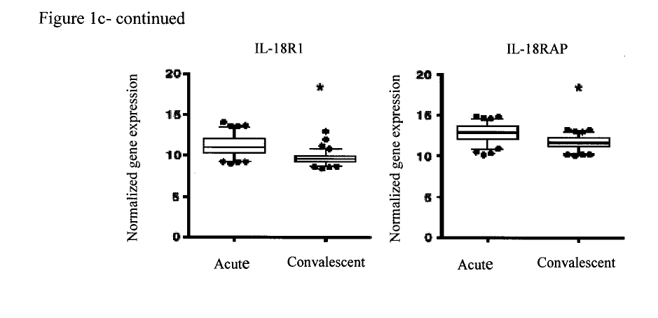

Circulating levels of IL1β and IL18 in acute KD. Children with KD have markedly elevated circulating protein levels of IL1β, IL18 and their respective antagonists, IL1 receptor antagonist (IL1RA) and IL18 binding protein (IL18BP), during the acute phase of KD, compared to age-matched febrile controls (with bacterial or viral infections) and their own convalescent phase controls (FIG. 1a). This correlates precisely with increased gene expression for their respective signaling receptor sub-units for both the IL1β and IL18 pathways (FIG. 1c).

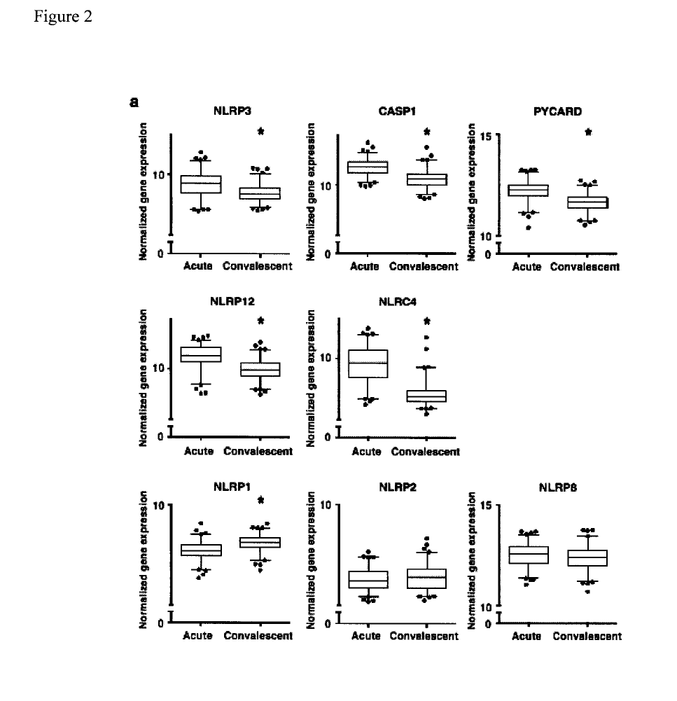

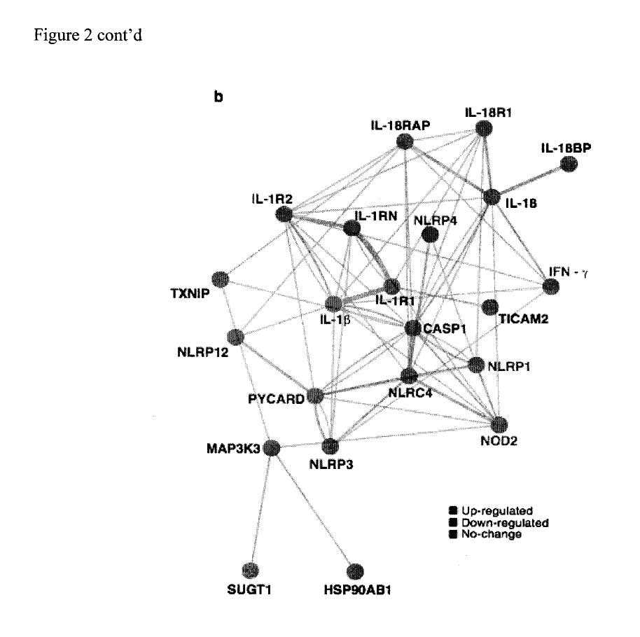

Expression of NLRP3 inflammasome associated genes during acute KD. Elevation of IL1β and IL18 is a signature associated with activation of the NLRP3 inflammasome; hence NLRP3 expression at the mRNA level was explored using gene expression data from serial samples (whole blood) obtained from children with KD (n=171). Up-regulation of NLRP3 mRNA was seen during the acute phase of KD and was accompanied by increased expression of Caspase-1 and PYCARD (FIG. 2a), key downstream effectors of inflammasome activation responsible for cleavage of pro-IL1β and pro-IL18 into their mature proteins. GeneMANIA (Warde-Farley et al. Nucleic Acids Research; 2010; 38 (Web Server):W214-W220) (www.genemania.org) was used to determine the relationship between various members of the IL1β/IL18 pathway, and showed that the increased gene expression profile is specific to NLRP3 inflammasome activation and observed only for molecules immediately downstream of NLRP3 activation and proteins also implicated in inflammasome activation including NLRC4 and NLRP12, but not unrelated NLRPs such as NLRP1, NLRP2, and NLRP8 (FIG. 2a-b).

ITPKC genotype dictates intracellular calcium mobilization. Generally, NLRP3 inflammasome activation is a two-step process with a priming signal and an activation signal, which is influenced by both extra- and intra-cellular calcium levels. ITPKC governs phosphorylation of IP3 to IP4 hence controlling calcium release from the endoplasmic reticulum. To determine the contribution of ITPKC in regulating [Ca^{2+]}i mobilization, bone marrow-derived macrophages (BMDM) from Itpkc-deficient and wildtype C57BL/6 control mice were stimulated and calcium flux was evaluated.

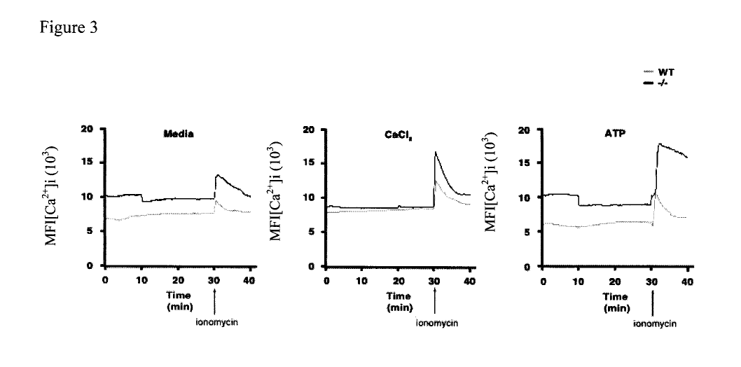

Baseline levels of [Ca²⁺]i were distinctly higher in the absence of ITPKC compared to wildtype controls, and stimulation with ionomycin resulted in a marked increase in [Ca²⁺]i quantified using Fluo-4AM by spinning disc confocal microscopy (FIG. 3). The addition of CaCl₂ to the culture medium increased basal [Ca²⁺]i in wild-type BMDM to match that of the ITPKC-deficient cells, but not calcium flux upon stimulation, which remained dramatically higher in the ITPKC-knockout cells. The difference in stimulated calcium flux was even more dramatic in the presence of ATP (FIG. 3). Therefore, ITPKC regulation of [Ca²⁺]i mobilization is independent of and additional to that of ATP's effect on release of [Ca²⁺]i.

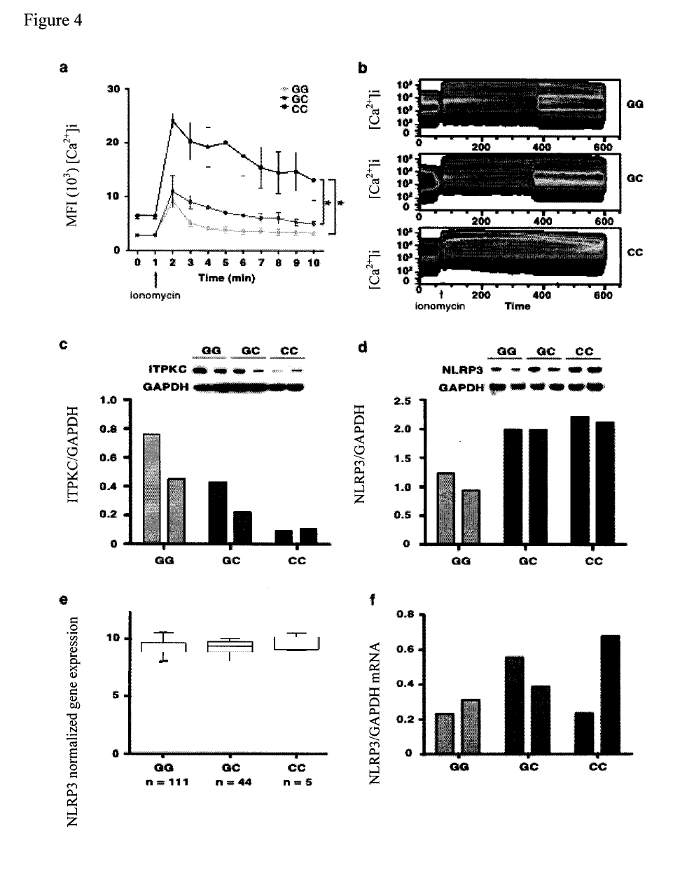

Next it was determined whether or not the ITPKC genotype associated with KD exhibited phenotypic and functional differences. Children with KD were genotyped at rs28493229, the ITPKC SNP for which the C allele is associated with KD. EBV immortalized B-cells from affected children were used in functional assays, where calcium flux was evaluated by flow cytometry using ratio-metrically opposite Ca²⁺ indicator dyes Fluo-4/AM and Fura-3AM in Ca²⁺ free media. Similar to the ITPKC-deficient murine cells, basal levels of [Ca²⁺]i were higher in cells from children with the CC genotype, which showed a marked increase in [Ca²⁺]i upon stimulation with ionomycin compared to cell lines from KD subjects carrying the GG and GC alleles (FIG. 4a-b). The kinetics of calcium mobilization were also distinct; upon stimulation, increased [Ca²⁺]i was sustained in duration in cells from children with the CC genotype compared to those with GG and GC alleles (FIG. 4a-b). The delay in return to basal levels was not complete at 10 minutes, the maximum length of time used for data capture by flow cytometry. Children with the CC genotype are infrequent, accounting for only 2.9% in a cohort of KD children of various ethnicities from the United States (n=171); which reflects the 1.5 fold increased frequency compared to the general population observed in the genetic discovery analysis. Given the small numbers of available EBV transformed B-cell lines from KD children with the CC genotype, the findings were confirmed in healthy controls with the disease-associated ITPKC genotype. Control adult EBV transformed cell lines were obtained from a healthy subject repository (TCAG, Toronto, Ontario, Canada). From 386 healthy individuals, 7 carried the CC allele (1.8%).

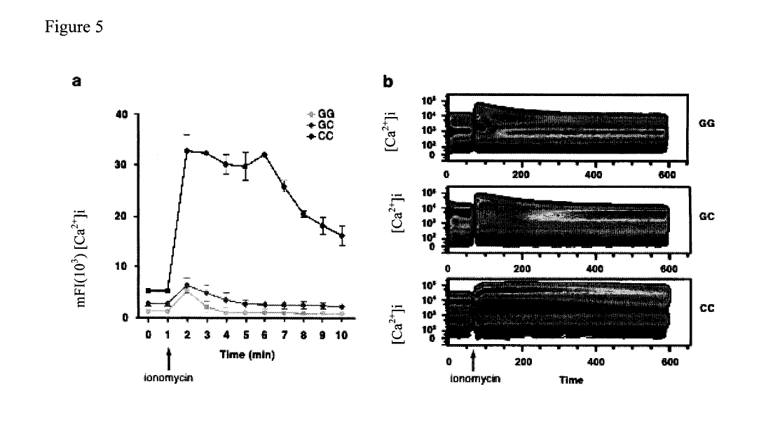

Results for calcium flux experiments performed on EBV immortalized B-cell lines from these 7 individuals were identical to those from the children with KD, showing the same phenotype, with increased basal levels of [Ca²⁺]i together with an exaggerated and prolonged increase upon stimulation (FIG. 5), thus the ITPKC genotype associated with KD has important functional consequences, governing [Ca²⁺]i both at baseline and in response to stimulation.

ITPKC genotype determines NLRP3 expression. The disease-associated polymorphism is located in an intron between exons 1 and 2 in the ITPKC gene. The substitution of C for G at position 8 nucleotide from the splice site is hypothesized to affect splicing efficiency with lower transcript abundance associated with the C allele. The functional differences seen in calcium mobilization between the different ITPKC genotypes paralleled expression of ITPKC protein (FIG. 4c). The association is striking—those with the disease-associated CC genotype had decreased ITPKC protein and increased amounts of NLRP3 protein compared to other genotypes at baseline (FIG. 4d). There was no difference in PYCARD protein expression between the genotypes (data not shown). The expression of NLRP3 was tightly associated with that of ITPKC protein, showing an inverse relationship (FIG. 4c-d), which is seen both at the NLRP3 mRNA and protein levels (FIG. 4f). The gene expression profile from children with acute KD show a trend towards increased expression of NLRP3 in those with the CC genotype, despite the small number of patients (FIG. 4e). The same was true in Itpkc-deficient mice.

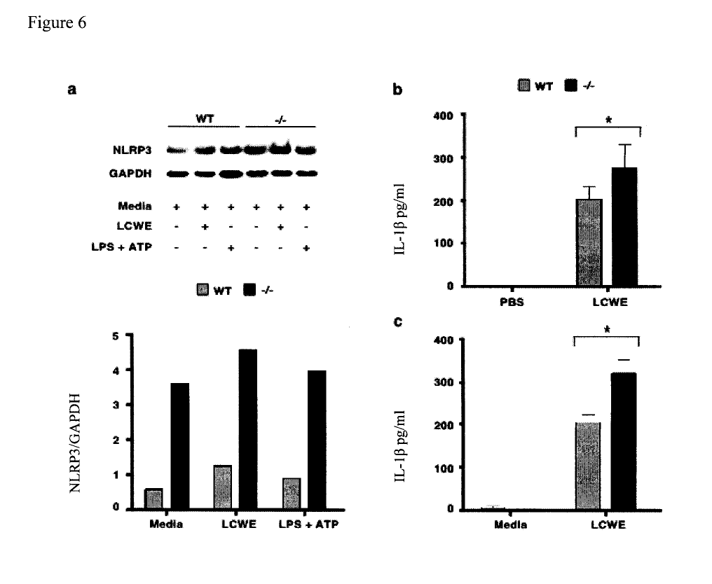

Indeed, BMDM from Itpkc-knockout mice had higher levels of NLRP3 at baseline (FIG. 6a). Lactobacillus casei cell wall extract (LCWE)-induced coronary arteritis in mice is an animal model for KD. It reflects human KD in its time course, pathology, susceptibility in the young and response to IVIG therapy. Itpkc-deficient mice showed increased NLRP3 expression upon LCWE-stimulation compared to wildtype C57BL/6 controls (FIG. 6a).

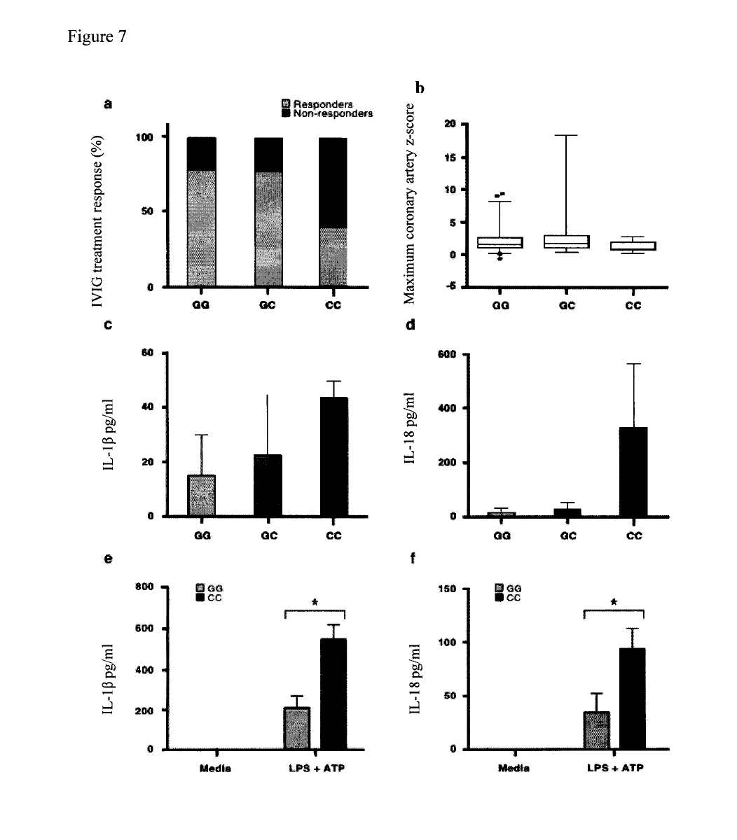

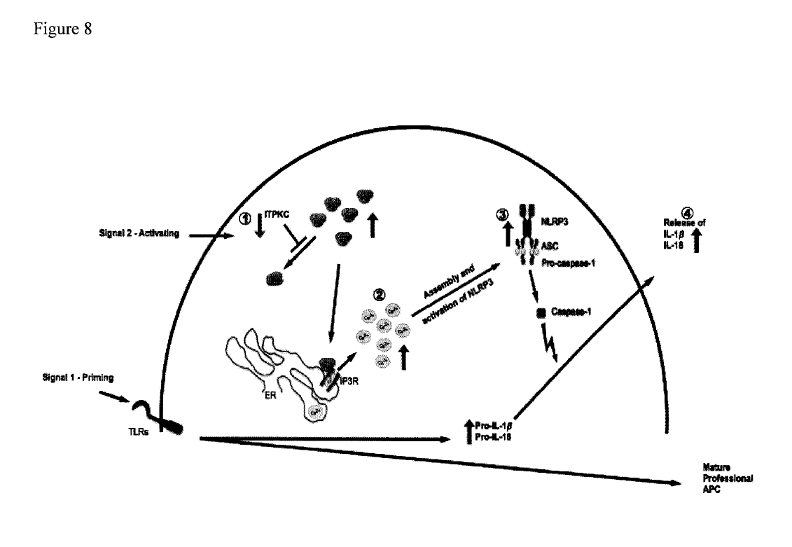

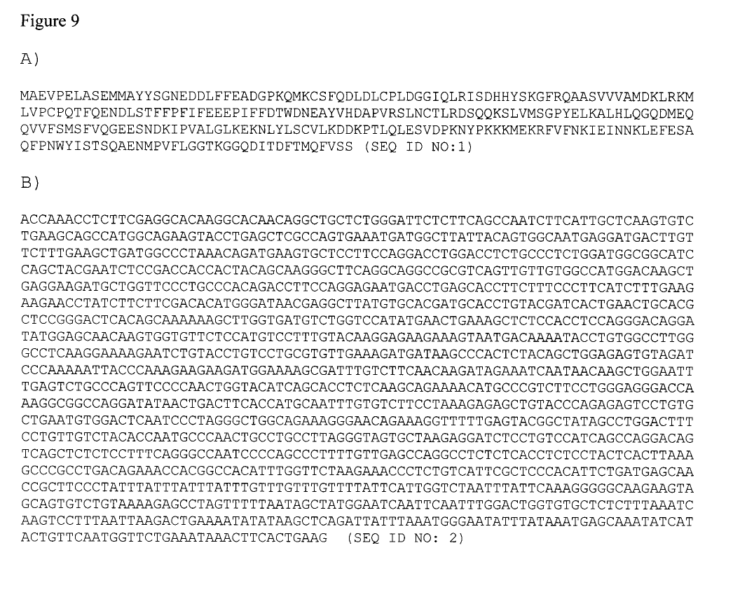

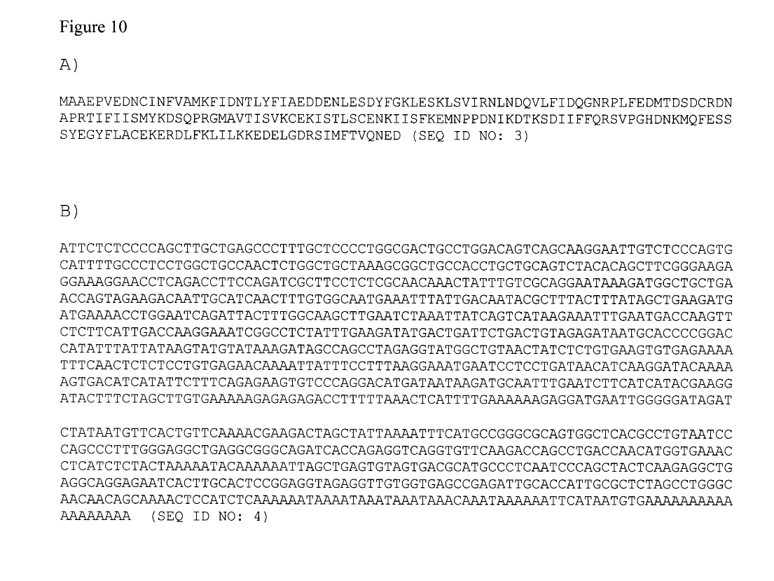

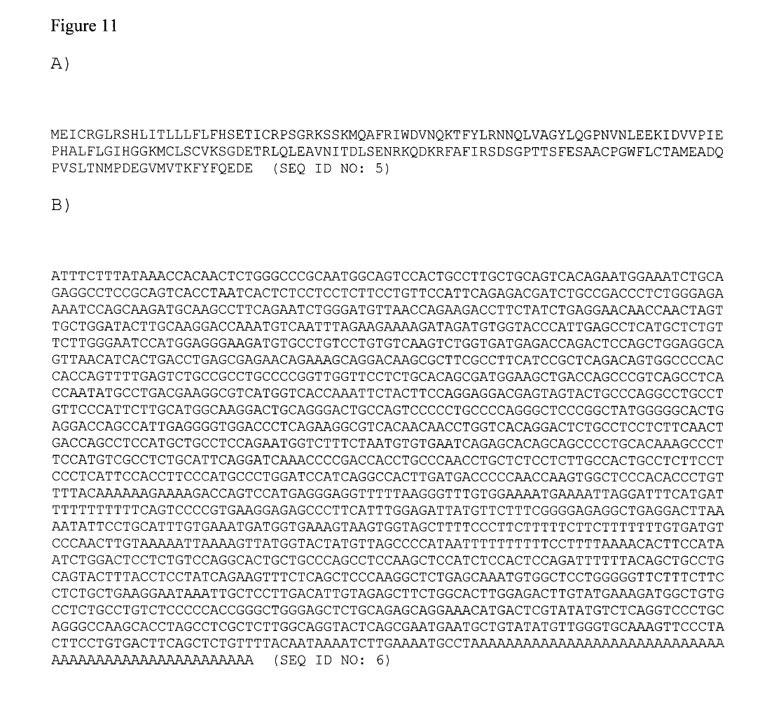

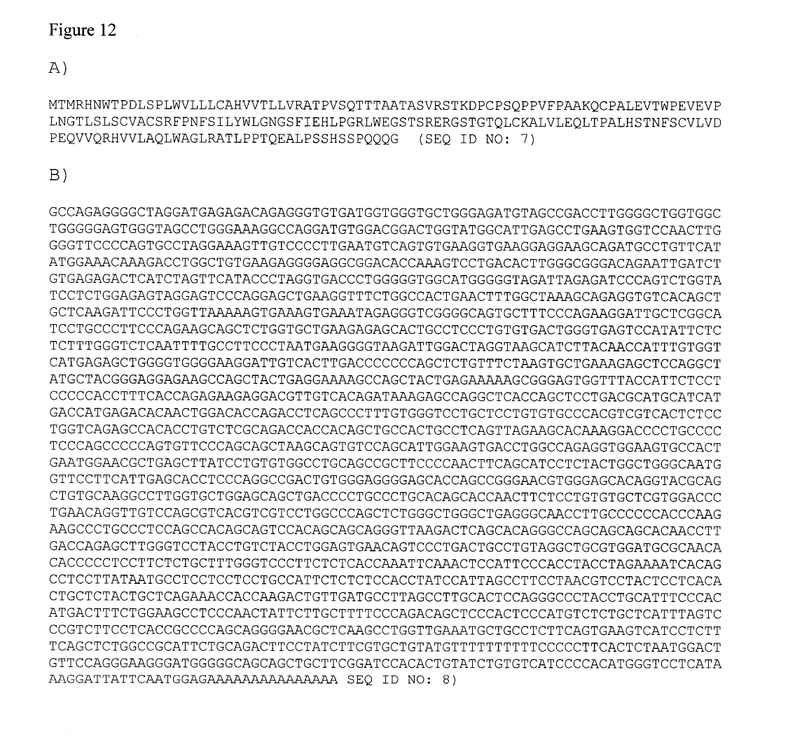

ITPKC directs treatment response and production of IL1β and IL18 in children with KD. The functional differences observed in calcium mobilization and NLRP3 activation for the 3 different ITPKC genotypes translated into substantial differences in treatment response. Intravenous immunoglobulin (IVIG) is the standard of care for the treatment of acute KD. Resistance to IVIG treatment is failure of primary treatment and was defined clinically as persistent or recrudescent fever 36 hours after completion of the IVIG infusion. IVIG non-response is correlated with severe disease and poor coronary outcome. Historically, the IVIG resistance rate is approximately 20% regardless of the cohort studied. In the present combined cohort of children with KD, the IVIG resistance rate was 22% in those with the GG or GC alleles compared to 60% in children with the CC genotype (FIG. 7a). Coronary outcome, as measured by development of coronary artery aneurysms and expressed as body surface area normalized z-scores for the affected children did not show a significant difference between the genotypes, although power was limited due to the small sample size (FIG. 7b). In the current era of IVIG treatment, coronary artery aneurysms occur in 5% of appropriately treated children, thus large numbers of subjects with each genotype would be required to detect significant differences in coronary artery outcome.

Circulating levels of IL1β and IL18 measured from plasma of affected children during the acute phase of KD also showed a striking correlation with ITPKC genotype—children with the CC genotype produced the highest concentrations of both cytokines (FIG. 7c-d). Functional testing of PBMCs obtained from affected children also point to a marked difference in biologic response between the ITPKC genotypes, with PBMCs from those with the CC genotype expressing significantly higher amounts of IL1β and IL18 upon stimulation with LPS and ATP, a prototypic combination of signals for NLRP3 inflammasome activation (FIG. 7e-f) (9). The same was observed in Itpkc-deficient mice.

Stimulation of mouse BMDMs with LCWE resulted in NLRP3 activation and exaggerated release of IL1β and IL18 in in vitro culture in Itpkc-deficient cells compared to those from C57BL/6-wildtype controls. Similarly, during disease development in the animal model, Itpkc-knock out mice produced higher levels of circulating IL1β and IL18 compared to wildtype controls (FIG. 6b-c).

1. A method of diagnosing Kawasaki disease in a mammal comprising determining in a biological sample obtained from the mammal the expression level of at least IL-1RA, and optionally one or more of IL-1β, IL-18 and IL-18BP; comparing the expression level of IL-1RA, and optionally one or more of IL-1β, IL-18 and IL-18BP, to a febrile control value; and diagnosing the mammal with Kawasaki disease when the expression level of IL-1RA, and optionally the expression level of one or more of IL-1β, IL-18 and IL-18BP, is greater than the febrile control value.

2. The method of claim 1, wherein the method comprises the determination of at least one of IL-1β, IL-18 and IL-18BP for comparison against a febrile control value.

3. The method of claim 1, wherein the expression of levels of IL-1β, IL1RA, IL-18 and IL-18BP are determined by an enzyme-linked immunosorbent assay.

4. The method of claim 1, wherein the biological sample is selected from the group consisting of blood, serum, plasma, urine, cerebrospinal fluid, ascitic fluid, lacrimal fluid, bone marrow or derivatives of any of these.

5. The method of claim 1, wherein the related protein is a receptor.

6. The method of claim 1, wherein the related protein for IL-1β is one of IL1R1 and IL-1R2.

7. The method of claim 1, wherein the related protein for IL-18 is one of IL18R1 and IL-18RAP.

8. The method of claim 1, wherein the level of IL-1RA is at least about 470 pg/ml, and optionally, the level of IL-1β is at least about 5 pg/ml, the level of IL-18 is at least about 115 pg/ml, and/or the level of IL-18BP is at least about 5 ng/ml.

9. A method of predicting treatment response phenotype in a mammal with Kawasaki disease, comprising determining in a biological sample obtained from the mammal the expression levels of IL-1β or a related protein, and IL-18 or a related protein, and determining that the mammal is at risk of being a non-responder to IVIG therapy when the level of IL-1β is greater than about 30 pg/ml, and/or the level of IL-18 is greater than about 95 pg/ml.

10. The method of claim 9, wherein the expression of levels of IL-1β and IL-18 are determined by an enzyme-linked immunosorbent assay.

11. The method of claim 9, wherein the biological sample is selected from the group consisting of blood, serum, plasma, urine, cerebrospinal fluid, ascitic fluid, lacrimal fluid, bone marrow or derivatives of any of these.

12. The method of claim 9, wherein the related protein is a receptor.

13. The method of claim 9, wherein the related protein for IL-1β is one of IL-1R1 and IL-1R2.

14. The method of claim 9, wherein the related protein for IL-18 is one of IL-18R1 and IL-18RAP.

15. A kit useful to diagnose Kawasaki disease in a mammal comprising a biomarker-specific reactant for IL-1 receptor antagonist, and at least one of IL-1β, IL-18, and IL-18 binding protein.

16. The kit of claim 15, additionally comprising instructions which indicate amounts of IL-1β, IL-18, IL-1 receptor antagonist and IL-18 binding protein which are indicative of Kawasaki disease.

17. The kit of claim 15, wherein the biomarker-specific reactant for each of IL-1β, IL-18, IL-1 receptor antagonist and IL-18 binding protein is an antibody.

18. (canceled)

19. (canceled)

20. (canceled)

21. The method of claim 9, comprising stimulating the biological sample obtained from the mammal with lipopolysaccharides and adenosine triphosphate and determining that the mammal is at risk of being a non-responder to IVIG therapy when the level of IL-1β is greater than about 400 pg/ml, and/or the level of IL-18 is greater than about 50 pg/ml.
